(19) United States
(12) Patent Application Publication
(10) Pub. No.: US 2003/0072802 A1
(43) Pub. Date: Apr. 17, 2003

(54) Sustained release topiramate

(75) Inventor: Neal R. Cutler, Los Angeles, CA (US)

Correspondence Address: MEDLEN & CARROLL, LLP, 101 HOWARD STREET, SUITE 350, SAN FRANCISCO, CA, 94105, US

(73) Assignee: R.T. Alamo Ventures, Inc.

(21) Appl. No.: 09/975,270

(22) Filed: Oct. 11, 2001

(51) Int. Cl. A61K 9/48; A61K 9/52; A61K 31/35; A61K 31/7008

(52) U.S. Cl. 424/468; 424/457; 514/23; 514/459

(57) ABSTRACT

The present invention is an improvement in the treatment of mania and depression by administering topiramate in a sustained-release formulation. The sustained-release formulation of the present invention may also be co-administered with anti-psychotics and anti-depressants.

### FIELD OF THE INVENTION

The present invention relates to the use of a sustained-release formulation of topiramate for the treatment of mania, depression and bipolar disorder.

### BACKGROUND OF INVENTION

Manic depression involves wide mood alterations, with periods of both depression and mania. A person experiencing depression or mania may have intense mood swings and consequent changes in thinking and behavior. The term that is professionally used for this illness is Bipolar Affective Disorder. Bipolar means sharing two poles (high and low) and Affective Disorder means a disorder having to do with mood.

The cause of manic depression is not known. Manic depression can send a person plunging from a high state, where one may believe one has superhuman energy and abilities, into a pit of despair, where it may seem as if the only way out is suicide. It is estimated that 1% of the population will have a manic-depressive illness. The number of manic and depressive episodes varies greatly from person to person and most individuals experience “normal” periods between their manic and depressive episodes.

There is no real cure for mania or depression at present but, through the use of monitored medication programs, it is possible to smooth out and reduce the frequency of the highs and the lows, and in some cases episodes may be altogether prevented. Some factors that determine the type of treatment care are the nature of the symptoms, the number of previous episodes, the severity and duration of the illness and previous response to treatments.

One of the major problems with drug-based treatment for mania and depression seems to be the aggravation of the up and down cycles of bipolar disorder due to uneven compound release and dosing. Uneven compound dosing is often complicated by poor patient compliance with current dosing regimes.

Therefore, what is needed is a pharmaceutical treatment that allows for controlled release of a therapeutic compound suitable for the treatment of mania and depression that will aid in increasing patient compliance.

### SUMMARY OF INVENTION

The present invention relates to the use of topiramate via a sustained-release pharmaceutical formulation for the treatment of mania, depression and bipolar disorder.

With formulations currently in use, topiramate plasma concentrations peak at about 2 hours after a 200 mg dose and taper off with a half life of about 21 hours. The variation due to these kinetic properties may not be advantageous for some patients. This may be especially true for patients exhibiting symptoms of mania, depression and bi-polar disorder since one of the symptoms of these disorders are wildly fluctuating mood swings. In other words, uneven administration of topiramate may, in part at least, exacerbate the patient's symptoms even as it helps to diminish them.

Poor compliance with taking antipsychotics has been reported in up to fifty percent of people with psychotic disorders (Tatton and Creed, Negative symptoms of schizophrenia and compliance with medication, Schizophr. Bull. 27:149-155, 2001). Poor compliance by patients has been cited as a major reason for relapse in the treatment of these disorders (Moore, et al., Compliance and psychological reactance in schizophrenia, Br. J. Clin. Psycol. 39:287-295, 2000). When using topiramate, the repeated dosages necessary with the current formulations may make patient compliance difficult. Simplification of medicine-taking behavior has been shown to be successful in addressing patient compliance problems. The sustained-release formulations of topiramate of the present invention will simplify the patient's medicine-taking. They will need to be administered less frequently and, therefore, permit better patient compliance.

Formulations and methods of producing sustained-release pharmaceutical compositions are contemplated. These include, but are not limited to, microcapsule compositions, microgranule compositions and compositions to be used for transoral administration. Formulations may include, but are not limited to, capsules, caplets, tablets, rapid dissolve matrices which release slow dissolve microcapsules or microgranuals, etc.

In one embodiment, the present invention contemplates a sustained-release formulation comprising topiramate. The present invention further contemplates an embodiment wherein the sustained-release formulation of topiramate is an oral formulation selected from a group consisting of microcapsules, microgranules, tablets, capsules and transoral formulations. In another embodient, the formulation is a suppository.

In one embodiment, the present invention contemplates a method of treatment comprising: a) providing i) a patient displaying one or more symptoms of mania and ii) a sustained-release formulation comprising topiramate; b) administering said formulation to said patient under conditions such that said one or more symptoms of said mania is reduced. In another embodiment, said topiramate is co-administered with an anti-psychotic.

In one embodiment, the present invention further contemplates a method of treatment, comprising: a) providing, i) a patient exhibiting one or more symptoms of depression and ii) a sustained-release formulation comprising topiramate; b) administering said formulation to said patient under conditions such that said one or more symptoms of said depression is reduced. In another embodiment, said topiramate is co-administered with an anti-depressant.

In one embodiment, the present invention additionally contemplates a method of treatment, comprising: a) providing, i) a patient exhibiting one or more symptoms of bipolar disorder and ii) a sustained-release formulation comprising topiramate; b) administering said formulation to said patient under conditions such that said one or more symptoms of said bipolar disorder is reduced.

In one embodiment, the present invention contemplates the co-administration of topiramate with other anti-psychotic or anti-depression medications. Examples of other anti-psychotic medications that may be co-administered with topiramate include, but are not limited to, Clozapine (clozaril), Risperdal (risperidone), Seroquel (quetiapine), Stelazine (trifluoperazine), Thorazine (chlorpromazine), lithium, Haldol (haloperidol), chlorpromazine, Mellaril (thioridazine), Prozac (fluoxetine), Ritalin (methylphenadate), Valium (diazepam; benzodiazepine), Zoloft (sertraline) and Zyprexa (olanzapine). The anti-psychotic compounds may be administered simultaneously or sequentially with topiramate. The anti-psychotic medications may be administered in any manner consistant with their accepted therapeutic use. Examples of anti-depression medications that can be administered with topiramate are, but not limited to, Prozac (fluoxetine), Paxil (paroxetine HCl), Zoloft (sertraline HCl), and Wellbutrin (bupropion HCl).

### DEFINITIONS

“Topiramate” is shown in Structure 1 and encompasses a pharmaceutical acceptable derivative or salt thereof.

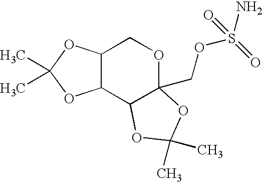

“One or more symptoms of mania” include excessive excitement, exalted feelings, delusions of grandeur, elevation of mood, psychomotor overreaction and overproduction of ideas.

“Depression” is a mental condition characterized by loss of interest in all usually pleasurable outlets such as food, sex, work, friends, hobbies or entertainment. Diagnostic criteria include presence of at least four of the following symptoms every day for at least two weeks: (1) poor appetite or significant weight loss or increased weight gain; (2) insomnia or hypersomnia; (3) psychomotor agitation or retardation; (4) loss of interest or pleasure in usual activities or decreased sex drive; (5) loss of energy or increased fatigue; (6) feelings of worthlessness, self-reproach or excessive or inappropriate guilt; (7) complaints of or evidence of diminished ability to think or concentrate; (8) recurrent thoughts of death, suicide ideation, wish to be dead or attempted suicide.

“Bipolar mania” or “bipolar disorder” is a disorder in which the patient exhibits both manic and depressive episodes.

“Sustained-release”, “delayed-release”, “controlled-release” and equivalent terms when used in the context of a formulation of a pharmaceutical composition indicates that the formulation releases the active ingredient over a length of time. Typically, the pharmaceutical composition is released over a period of about one to twelve hours. More typically, the pharmaceutical composition is released over a period of about four to eight hours. An oral formulation of this type is simply one that can be taken orally.

“Transoral” is a mode of pharmaceutical administration wherein the compound and/or delivery system is administered to the patient orally. Any form of oral administration is contemplated in the present invention. For example, chewing and swallowing may be included in the administration of the compound or the compound may be allowed to dissolve in the buccal cavity.

“Administered in combination”, “co-administered” or equivalent terms, shall be defined as pharmaceuticals that are administered simultaneously or sequentially with topiramate. The pharmaceuticals administered need not be in the same dosage form (e.g., sustained release) as the topiramate. “In combination with” topiramate shall be defined as the administration of the other drug either simultaneously or sequentially with topiramate.

“High insolubility,” when applied to pharmaceutical salts, shall be defined as pharmaceutical salts that dissolve in the gastro-intestinal (GI) environment at a rate equivalent to sustained release formulations of the pharmaceutical, e.g., from four to six hours.

“Mania-ameliorating amount”, “mania-alleviating amount”, “depression-ameliorating amount” or “depression-alleviating amount” shall be defined as an amount of topiramate which effects a prophylactic or therapeutic response in the patient in need of such a response, over a period of about one to twelve hours or four to eight hours, causing either a reduction or an eradication of one or more of the symptoms of mania or depression. Importantly, it is not intended that the present invention be limited to formulations or dosages which eliminate one of all symptoms. It is sufficient that one or more symptoms is reduced (e.g.) in severity, duration, number of occurrences over time, etc.

“Dosage form,” in accordance with the present invention, includes any formulation that permits the sustained-release of a pharmaceutical preparation of topiramate in a patient. Examples are sustained-release transoral formulations, compressed tablets, microcapsules and microgranules.

“Dissolvable,” shall be defined as describing the action of the dosage form as it is held in the mouth, digestive track or bowels. In this case, the dosage form gets continuously smaller in a process that is conceptually analogous to melting. The dosage form may also disintegrate into smaller pieces to some extent, particularly where a relatively greater amount of a wicking agent or effervescent disintegrate is used, or where the dosage form is chewed. In another embodiment, dissolvable shall be defined as the release of a pharmaceutical agent from a matrix, microcapsule, microgranule, transdermal patch, etc., or aiding in regulating dissolve rate or release rate.

“Water soluble” shall be defined as being dissolvable in water or other aqueous solution. The dissolving may be rapid or slow depending of the substance being dissolved. For example, a coated pharmaceutical microcapsule may dissolve rapidly if coated with sugar or slowly if coated with a cellulose polymer. Sustained-release pharmaceutical formulations may provide controlled dissolution in an aqueous solution.

“Oil soluble” shall be defined as being readily dissolvable in an organic or non-organic fat-, lipid-, or oil-based (etc.) solution.

“Suspension” shall be defined as a state of a solid when its particles are mixed with, but not dissolved in, a fluid.

“Coacervate” shall be defined as the formation of an aggregate in a solution or suspension.

“Therapeutic formulation” shall be described as a pharmaceutical composition comprising at least one active ingredient along with other optional ingredients useful in, for example, binding, flavoring, coloring, preserving, stabilizing, increasing self life, adding structural rigidity, adding desired mouth feel, adding desired mouth consistency, providing ease of manufacturing, aiding in regulating dissolve rate, adjusting the pH of the local environment.

“Reduced” and “reduced symptoms” shall be defined as a lessening of symptoms to a noticeable degree by either the subject or medical professional. In the context of the present invention reduced symptoms shall mean, for example, the lessened severity of the subject's bipolar disorder. “Lessened severity” shall be defined, for example, as a lessening of mood swings, reduced thoughts of depression or suicide, reduced feeling of euphoria, an increased ability for the subject to perform his or her normal routine, etc. It is not necessary, in the context of the present invention, for the treatment completely to relieve or eliminate all symptoms (e.g. such as the symptoms of bipolar disorder). It is sufficient that one or more symptoms may be reduced.

### GENERAL DESCRIPTION OF INVENTION

In one embodiment, the present invention relates to the use of a sustained-release formulation of topiramate for the treatment of mania. The cause of mania is not known. Any person can develop manic depression, however, studies indicate that highly creative, sensitive people, people tending to be perfectionists and high achievers, have a higher incidence of Bipolar Affective Disorder. Although biological factors seem to play a major role in producing the illness, a person's personality make up and or stresses in the environment may also play a part in bringing on an acute episode.

Manic depression can send a person plunging from a high state, where one may believe one has superhuman energy and abilities, into a pit of despair, where it may seem as if the only way out is suicide. The common symptoms of the depressive state include a sad, despairing mood, which may be accompanied by some or all of the following: lack of energy, sleep problems where a person may sleep too much or too little, loss of interest in work, family and friends, change in eating habits, preoccupation with failures and inadequacies, loss of self-esteem, feelings of guilt, excessive concern about physical complaints, decreased sexual drive, crying easily and suicidal and occasional homicidal thoughts.

The symptoms of the manic state include the feeling of total “euphoria” and strength. However, in the early stages of the illness, the person may appear to be more sociable, active, talkative, self-confident, perceptive, and creative than usual. As his/her mood elevates, he may experience some or all of the following: increased strength and energy, decreased sleep, extreme irritability, rapid, unpredictable emotional changes, racing thoughts, flight of ideas, increased interest in activities, overspending, grandiosity, inflated self-esteem, increased sexual drive and poor judgment.

### DETAILED DESCRIPTION OF PREFERRED EMBODIMENTS

In certain embodiments, the present invention is directed, among other things, to methods of treating mania and depression by the sustained-release administration of a mania-ameliorating or alleviating or depression-ameliorating or alleviating effective amount of topiramate or an effective pharmaceutical derivative or salt thereof alone or in combination with other psycotropics. In one embodiment of the present invention, the one or more symptoms of mania or depression are reduced after administration of a sustained-release dose of topiramate.

Although embodiments of the present invention are not limited to any particular mechanism, many physiological factors influence the release of a drug from a controlled release dosage form and, thus, influence the uptake of the drug into the systemic circulation. Dosage forms should therefore be designed so that such variable factors do not compromise the efficacy and safety of the product. Ideally, such sustained-release dosage forms should release the active pharmaceutical ingredient at a controlled rate such that the amount of active pharmaceutical ingredient which is available in the body to treat the condition is maintained at a relatively constant level over an extended period of time. That is, it is desirable that active pharmaceutical ingredient be released at a reproducible, predictable rate which is substantially independent of physiological factors which can vary considerably among different individuals and even over time for a particular individual.

The precise mechanism by which the sustained-release administration of a mania-ameliorating or depression-ameliorating effective amount of topiramate relieves mania or depression is unknown. However, without limiting any embodiment of the invention to any particular theory, three properties may contribute to topiramate's antimania and antidepression, efficacy. First, in cultured neurons, action potentials elicited repetitively by a sustained depolarization of the neurons are blocked by topiramate in a time-dependent manner, suggestive of a state-dependent sodium channel blocking action. Second, topiramate increases the frequency at which γ-aminobutyrate (GABA) activates GABA_{A} receptors, and enhances the ability of GABA to induce a flux of chloride ions into neurons, suggesting that topiramate potentates the activity of this inhibitory neurotransmitter. This effect was not blocked by flumazenil, a benzodiazepine antagonist, nor did topiramate increase the duration of the channel open time, differentiating topiramate from barbiturates that modulate GABA_{A} receptors. Third, topiramate antagonizes the ability of kainate to activate the kainate/AMPA (-amino-3-hydroxy-5-methylisoxazole-4-propionic acid; non-NMDA) subtype of excitatory amino acid (glutamate) receptor, but has no apparent effect on the activity of N-methyl-D-aspartate (NMDA) at the NMDA receptor subtype. These effects of topiramate are concentration-dependent within the range of 1 μM to 200 μM. Although not limiting any embodiment of the present invention, these findings suggest concentration dependent mechanisms at the receptor site which may not always be suggested by the absolute plasma concentration.

Production of Topiramate

Topiramate can be prepared following the processes disclosed in U.S. Pat. Nos. 4,513,006 and 5,387,700, and preferably, by the process described in Examples 1 to 3 of U.S. Pat. No. 5,387,700 all of which are hereby incorporated by reference. Topiramate is one of a variety of chlorosulfate and sulfamate esters of 2,3:4,5-bis-Q-(1-methylethylidene)+-D-fructopyranose. Their anticonvulsant activity is known in mammals and, thus, their utility in treating diseases such as epilepsy and glaucoma, are described in U.S. Pat. No. 4,513,006 hereby incorporated by reference. More specifically, the compound 2,3:4,5-bis-Q-(1-methylethylidene)-p-D-fructopyranose sulfamate, hereinafter referred to as “topiramate”, is presently available for marketing as a tablet product in strengths of 25, 50, 100, 200, 300 and 400 mg as adjunctive therapy for the treatment of adults with partial onset seizures (TOPAMAXB™ (topiramate) tablets). Topiramate is also available in a “sprinkle” formulation for the ability to add the drug to foods. Degradation of topiramate is readily detected by changes in physical appearance, i.e., discoloration to brown or black, and by the formation of sulfate ions which can be readily detected by standard techniques know to those of ordinary skill in the art (e.g., HPLC).

In one embodiment of the present invention it is contemplated that topiramate is combined with inactive ingredients. Such ingredients may be necessary, e.g. to add bulk to the pharmaceutical preparation, to bind the preparation, to add color or flavor to the preparation, to prevent degradation or growth of contaminants, to aid in the ease of manufacturing, etc.

Sustained-Release Pharmaceutical Formulations

This section provides embodiments of several representative sustained-release pharmaceutical formulations of topiramate. This section is not intended to be exhaustive and not intended to limit any embodiment of the present invention in any way. Other methods of producing and administering sustained-release topiramate may be employed.

The suitability of administration of topiramate, its method of administration, dosage, and timing of administration will be apparent to a medical professional and dependent on the patient and the nature and severity of symptoms. In most instances dosing and administration will be determined by a medical practitioner at the time of and subsequent to diagnosis. In one embodiment, it is contemplated that the dose of topiramate will be between 15 mg and 1500 mg per day. In another embodiment, it is contemplated that the dose of topiramate will be between 50 and 800 mg per day. In a more preferred embodiment, it is contemplated that the dose of topiramate will be between 100 and 500 mg per day.

1. Transoral

In one embodiment of the present invention, it is contemplated that there is a sustained release composition of topiramate comprising a controlled release hydrophilic matrix system. Although the method by which the matrix system controls the release of pharmaceuticals in no way limits any embodiment of the present invention, it is believed that hydroxypropyl methylcellulose works by the outer surface of the tablet, caplet, etc. hydrating. Water permeates into the tablet, increasing the thickness of the outer layer causing it to become a gel and thereby limiting the escape of the pharmaceutical from the tablet.

In one embodiment, it is contemplated that the matrix system comprises hydroxypropyl methylcellulose. In another embodiment it is contemplated that the matrix system comprises Methocel™ (DOW, Edison, N.J.) hydroxypropyl methylcellulose. In a preferred embodiment, the matrix system comprises Methocel™ E, F or K hydroxypropyl methylcellulose. It will be noted that one practiced in the art may substitute other brands of hydroxypropyl methocellulose for Methocel™. The production of hydroxypropyl methocellulose based sustained-release and controlled-release pharmaceuticals is known to those practiced in the art (Using METHOCEL Cellulose Ethers for Controlled Release of Drugs in Hydrophilic Matrix Systems, DOW, Edison, N.J.). Additional examples of sustained-release formulations comprising hydroxypropyl methocellulose matrices are given in U.S. Pat. No. 4,389,393 to Schor, et al., which is incorporated herein by reference.

In another embodiment of the present invention, the matrix system is designed to rapidly disintegrate. Topiramate is contained in the matrix in sustained-release coated microgranuals or coated microcapsules. Upon disintegration, the pharmaceutical (e.g., topiramate) is released from the matrix. Topiramate is slowly released from the coated microgranuale or capsules.

In a another embodiment, it is contemplated that the present invention provides a hard, compressed, rapidly dissolving, controlled release tablet adapted for transoral dosing. The tablet includes particles made of an active ingredient and a protective material. These particles are provided in an amount of between about 0.01 and about 75% by weight based on the weight of the tablet. The tablet also includes a matrix made from a nondirect compression filler, a wicking agent, and a hydrophobic lubricant. The tablet matrix comprises at least about 60% rapidly water-soluble ingredients based on the total weight of the matrix material. The tablet has a hardness of between about 15 and about 50 Newtons, a friability of less than 2% when measured by U.S.P. and is adapted to dissolve slowly in the mouth of a patient in more than about 1 hour and thereby liberate said particles and be capable of being stored in bulk.

In one embodiment of the present invention it is contemplated that topiramate is combined with inactive ingredients. Such ingredients may be necessary, e.g., to add bulk to the pharmaceutical preparation, to bind the preparation, to add color or flavor to the preparation, to prevent degradation or growth of contaminants, aid in the ease of manufacturing, etc.

2. Compressed Tablets

In another preferred embodiment, the oral formulation of topiramate comprises a hard or compressed powder tablet designed to dissolve in the stomach or digestive track in about 2 to 12 hours. Methods of producing sustained-release tablets are disclosed in U.S. Pat. Nos. 5,164,193; 6,126,969 and 6,221,392; all of which are incorporated herein by reference.

Although the present invention is not limited to any particular theory, the release of an active pharmaceutical ingredient from a controlled-release or sustained-release dosage form is generally controlled either by diffusion through a coating, by diffusion of the agent from a monolithic device, or by erosion of a coating by a process which is dependent upon enzymes or pH.

In accordance with one embodiment of the invention, a sustained-release topiramate formulation is provided. The topiramate is preferably provided in a finally divided form such as small particles or granules. The topiramate particles preferably have an average particle size between about 180 microns to 425 microns. The topiramate particles may contain excipients, adjuvants or other active ingredients in minor amounts if desired, but are more preferably comprised of at least 10, 25, 50, 75, 90 or 95% topiramate.

The sustained-release topiramate formulations, in accordance with the first aspect of the invention, are provided with a sustained-release coating formulation comprising a water-insoluble, water-permeable and slightly water-swellable polymer coating. The polymeric coating is not soluble in the gastrointestinal fluids and is not sensitive to the pH thereof. The term “pH independent” as used herein means that the water permeability of the coating, and hence its ability to release pharmaceutical ingredients, is not a function of pH or is only very slightly dependent on pH. Accordingly, the sustained-release topiramate formulations of the present invention are capable of releasing topiramate into the gastrointestinal tract at a controlled rate which is independent of physiological factors such as pH, which can vary from one subject to another and can vary from time to time for a particular subject.

Although not limited to any particular formula, in one embodiment the polymeric coating preferably comprises a methacrylate ester copolymer having the general formula:

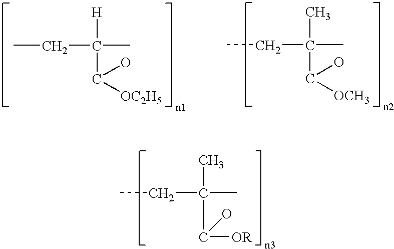

wherein R is an alkyl or aminoalkyl group having from 1 to about 12 carbon atoms, and wherein n₁, n₂, and n₃ are selected so that the polymer has a molecular weight of from about 100,000 to about 1,000,000. The ratio n₁:n₂ is from about 1:2 to about 2:1, and the ratio of n₃: (n.₁+n₂) is from about 0 to about 1:15. Particularly preferred are copolymers of ethylacrylate and methylmethacrylate, although terpolymers and other polymers comprising three or more different monomeric units having property similar to ethylacrylate-methylenethacrylate copolymers are also preferred. The preferred methacrylate-ethylacrylate copolymer has a molecular weight of approximately 800,000. A particularly preferred methylmethacrylate-ethylacrylate copolymer is Eudragrit™ NE30D which is commercially available from Rohm Pharma.

The polymeric film coating can be applied to the topiramate particles in any suitable manner. Preferably, the polymeric film is applied as a uniform coating having a smooth surface structure and a relatively constant thickness. A particularly preferred method of applying the polymeric coating to the topiramate particles is by utilizing pneumatic spray guns. The pneumatic spray guns preferably have a nozzle diameter of from about 0.8 mm to about 2 mm, and are operated at an air pressure of from about 0.5 to about 3 bar. The spraying rate of the spray guns can be easily regulated using peristaltic pumps or pressure vessels. Ideally, spraying should be continuous with simultaneous drying, so that the particles do not become too moist (over wet). The freshly sprayed polymeric film should dry as quickly as possible to avoid a agglomeration of the particles. Fluidized-bed processes are particularly suitable for coating small particles. For example fluidized-bed systems such as Aeromatic, Glatt with Wurster HS Column, operate in closed cylindrical apparatuses into which an air stream is introduced from below to fluidized the topiramate particles and dry the films during spraying. In addition to fluidized-bed processes, modified coating drums (usually cylindrical horizontally rotating units with a perforated wall) are also suitable for coating small particles.

The topiramate particles having a polymeric controlled release coating can be further manufactured into various types of oral dosage forms. For example, the release coated topiramate particles can be compressed, either alone or in combination with excipients, adjuvants and/or other active ingredients, into pills, tablets or the like. As another example, the release coated particles can be loaded into capsules such as either soft gelatin capsules or hard gelatin capsules. As another example, the release coated particles can be packaged into a pouch with other active or inactive ingredients. It can be dispersed into water in form of suspension.

The coating composition may include minor amounts of emulsifiers, wetting agents and stabilizers such as isononylphenylpolyoxethylene glycol ethers. Minor amounts of talc can also be incorporated into the coating composition or subsequently applied to improve or enhance the flow properties of the coated particles.

Suitable coating thicknesses can range from about 2 micrometers to about 15 micrometers, depending on the desired diffusion properties. The weight of the coating is generally between 2 and 50% of the weight of the topiramate particles.

In accordance with another embodiment of the invention, the release coated topiramate particles can be combined with uncoated topiramate particles to provide an orally administrable pharmaceutical formulation having both immediate-release and sustained-release components. The uncoated topiramate particles may generally have substantially the same characteristics as the release coated particles prior to coating. As with the release coated particles, the uncoated particles may contain minor amounts of excipients, adjuvants and/or other active ingredients.

In the broader aspects of the invention, can be substituted for a variety of other release-coatings, including soluble, insoluble, permeable, impermeable or bio-degradable coatings, the water-insoluble, water-permeable and water-swellable polymer coatings previously set forth. The polymer coating can be comprised of one or more polymers, including copolymers, terpolymers and other polymers having three or more different monomeric units. The polymers may include natural or synthetic polymers. Natural polymers which may be utilized in the sustained-release coating include polypeptides, polysacarides and alginic acid. Representative synthetic polymers include aqueous cellulose, hydroxyacyl cellulose, cellulose ether, cellulose esters, nitrocellulose, polymers of acrylic and methacrylic acids and esters thereof, polyamides, polycarbonates, polyalkylenes, polyalkylene glycol, polyalkylene oxides, polyalkylene terephthalates, polyvinyl alcohols, polyvinyl ethers, polyvinyl esters, polyvinyl halides, polyvinyl pyrrolidone, polyglycolides, polysiloxanes and polyurethanes and copolymers thereof.

Particular examples of suitable polymers for use in the sustained-release coating of the combined immediate-release/sustained-release topiramate formulations includes: methyl cellulose, ethyl cellulose, hydroxypropyl cellulose, hydroxypropylmethyl cellulose (e.g., Methocel™), hydroxybutylmethyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, polymethylmethacrylate, polyethylmethacrylate, polybutymethacrylate, polyisobutymethacrylate, polyhexomethacrylate, polyisodecylmethacrylate, poly(lauryl methacrylate), poly(phenyl methacrylate), polymethalacrylate, polyisopropylacrylate, polyisbutalacrylate, polyoctadcylacrylate, polyethylene (low or high density), polypropolyne, polyethylene glycol, polyethylene oxide, polyethylene terephthalate, polyvinyl alcohol, polyvinyl isobutyl ether, polyvinyl acetate, polyvinyl chloride and polyvinyl pyrrolidone. Examples of suitable copolymers include: butylmethacrylate/isobutylmethacrylate copolymer, high molecular weight, methylvinyl ether/maleic acid copolymer, methlvinyl ether/maleic acid, monoethyl ester copolymer, methylvinyl ether/malec and anhydride copolymer and vinyl alcohol/vinyl acetate copolymer. Examples of suitable biodegradable polymers include: polylactides, polyglycolides, polyethylene terathatic and polyeurathine. Examples of suitable acrylate and methacrylate are polyacrylic and methacrylic polymer such as those sold under the trademark Eudragit™.

The coated topiramate particles can be combined in various oral pharmaceutical formulations such as capsules, tablets, pouches or the like. The sustained-release coated topiramate particles can be combined with various excipients, adjuvants, and/or other active ingredients compressed into a tablet.

Another embodiment of the invention includes particles of topiramate which are coated with a water-insoluble, water-permeable, and slightly water-swellable polymeric coating which provides diffusion controlled sustained-release of the active ingredient at a highly reproducible, predictable rate which is independent of inter- and intra-subject physiological variations such as pH, combined with an uncoated pharmaceutically active ingredient which can be the same or different from the sustained-release coated pharmaceutically active ingredient. The resulting combined immediate-release/sustained-release formulation provides higher reproducibility of drug release rates than other sustained-release dosage forms utilizing conventional enteric sustained-release coating compositions, while providing both immediate and sustained-release of medicaments.

While any of the above embodiments of the invention can be formulated into generally any of a variety of different types of orally administrable pharmaceutical dosage forms such as capsules, the pharmaceutical compositions of the invention are most preferably pressed into tablets. The tablets preferably have a hardness of from about 11 to about 19 SCU, and most preferably a hardness of about 15 SCU.

Suitable excipients and adjuvants which can be used in the preparation of the sustained-release therapeutic compositions of the present invention generally include those conventionally used in the pharmaceutical industry. Examples include fillers and diluents such lactose, sucrose, dextrose, mannitol, calcium sulphate, dicalcuim sulphate, tricalcuim sulphate, micro-crystalline cellulose and starches such as rice starch. Useful binders include acacia, tragacanth, gelatine, sucrose, pre-gelatinized starch, starch, sodium alginate, ammonium calcium alginate, methylcellulose, sodium carboxymethyl cellulose, ethel cellulose, hydroxypropylmethyl cellulose (e.g., Methocel™), polyvinylpyrrolidone, mechanism aluminum ciliate and polyacrylamide. Examples of disintigrants include cross-linked polyvinylpyrrolidone, starch derivatives such as carboxymethyl cellulose and cellulose derivatives. Lubricants, guidance and anti-adhesive agents include metallic stearates such as magnesium stearate, talc, high melting point waxes, and colordacylica.

For the various topiramate formulations disclosed herein, preferred combination disintigrants/binders include cross-linked polyvinylpyrrolidone such as Polyplasdone XL™ available from GAF or croscarmellose sodium such as Ac-Di-Sol™ available from FMC Corporation. Another disintigrants/binder which is preferably utilized in combination with the cross-linked polyvinylpyrrolidone or croscarmellose sodium is micro-crystalline cellulose. The preferred disintigrants/binders can be utilized in effective amounts which can be readily determined using known techniques. The compositions of the invention are typically directly tableted using a conventional tableting apparatus, e.g., a Manesty Rotary Press, a Stokes Rotary Press, etc., at a temperature of about 15.degree. to about 30° C. and at a pressure of from about 0.4 to about 3.0 tons.

The tablets are desirably provided with a coating which helps prevent dusting of the tablets during handling and in the bottle, and which improves the appearance and swallowability of the tablets. A preferred tablet coating is set forth in the examples. The coated tablets are also preferably provided with an overcoat of carnauba wax which provides the tablets with a glossy appearance.

3. Microcapsules and Microgranules

In one embodiment, the present invention contemplates sustained-release microcapsule and microgranule delivery of topiramate. This embodiment of the present invention is not limited to any particular method of microcapsule and microgranule delivery of topiramate. In a preferred embodiment, the methods of U.S. Pat. Nos. 4,894,239; 6,045,830 and 6,117,455 are contemplated and are all incorporated herein by reference.

In a preferred embodiment, the present invention contemplates a sustained-release microcapsule preparation comprising an ion exchange resin with 6 to 16% of crosslinking, containing topiramate adsorbed in an amount not less than 80% of its theoretical ion adsorption amount (the topiramate-resin complex), and coated with a water-permeable polymer coat, a method of producing the sustained-release microcapsule preparation comprising coating an ion exchange resin with 6 to 16% crosslinking and containing topiramate adsorbed in an amount not less than 80% of its theoretical ion adsorption amount with a water-permeable polymer.

As for the ion exchange resin forming the above mentioned topiramate-resin complex, ordinary synthetic insoluble porous polymers (e.g., the polymer which is the copolymer of styrene and divinylbenzene) may be mentioned.

The polymer is a basic ion exchange resin (OH type), it contains primary to quaternary amino groups, etc., and adsorbs topiramate. In the present invention, in particular, it is preferable to use a strongly alkaline ion exchange resin. The degree of crosslinking for the ion exchange resin is determined depending upon the amount of crosslinking agent such as divinylbenzene to be used; it is preferable that crosslinking is from 6 to 16%, especially from 8 to 14%.

These ion exchange resins are commercially available under the trade names of Diaion™ (Mitsubishi Chemical Industries Ltd., Japan), Dowex™ (Dow Chemical Co., USA), Amberlite™ (Rohm & Haas Co., USA), and others, and can be selected for use as appropriate.

It is preferable that the mean particle size of the ion exchange resin is from 5 to 1000 μm, specifically from 10 to 300 μm. If desired, a commercially available ion exchange resin may be crushed to fine particles before use by means of a mill such as an atomizer.

The theoretical ion adsorption amount (theoretical saturated adsorption amount, overall exchanging capacity) means the maximum amount of strongly basic or acidic ions adsorbed by a given ion exchange resin. For the present invention, an ion exchange resin which has adsorbed a drug in a molar ratio of more than 80%, specifically from 85 to 100% of this theoretical amount, is preferred.

The water-permeable polymer coat is formed of a natural or non-natural biocompatible polymer. Examples of such polymers, include cellulose polymers such as ethylcellulose, nitrocellulose, benzylcellulose, acetocellulose, hydroxypropylcellulose and cellulose acetate propionate; and non-natural polymers such as polyacrylate, polymethacrylate, polyamide and acrylate-methacrylate copolymers (e.g., aminoalkyl methacrylate copolymer). For the present invention, in particular, aminoalkyl methacrylates (known as Eudragit, etc.) are favored.

Although not limited to any particular method, the sustained-release preparation of this embodiment of this invention can be, for example, produced as follows: In an aqueous solvent capable of dissolving both salts and the free form of the drug (i.e., topiramate), a drug in a salt form is reacted with an ion exchange resin to give an aqueous solution of the free form of the drug.

Examples of aqueous solvents include organic solvents which are freely soluble in water such as primary, lower (C₁₋₃) alcohols (e.g., methanol, ethanol, isopropanol) and aqueous solutions of ketones such as acetone and methyl ethyl ketone.

Said aqueous solvents mentioned include aqueous solutions containing from 5 to 95%, preferably from 10 to 90%, of the organic solvent. In particular, when the salt is of an acidic drug, to use an aqueous solution of from 5 to 30% ethanol.

The ion exchange resin to be used in the reaction may be those mentioned above, i.e., a basic ion exchange resin is used for a basic drug and an acidic ion exchange is used for an acidic drug to make respective free forms.

The reaction with the ion exchange resin is carried out by adding an ion exchange resin as mentioned above to the salt of the drug (i.e., topiramate) in solution in the aqueous solvent and then stirring the mixture. In this case, it is preferable that the ion exchange resin is used in an amount from 1.0 to 2.0 times the necessary amount of the drug's salt. The reaction is normally carried out at room temperature or ambient temperature, but the mixture can be warmed to about 70° C. Reaction time is from 0.5 to 6 hours. After the completion of reaction, the ion exchange resin is removed by ordinary means, then an aqueous solution of the free form of the drug is provided.

The topiramate-resin complex can be produced by adding an ion exchange resin of a given particle size and degree of crosslinking to the above aqueous solution and causing a reaction between them. The reaction is normally carried out at room temperature with from 0.5 to 3 hours of stirring.

The reaction will give a topiramate-ion exchange resin complex which has adsorbed the drug in an amount of more than 80% of the theoretical ion adsorption amount, and it is preferable that the complex used contain the adsorbed drug in an amount of from 85 to 100% of the theoretical ion adsorption amount.

The complex is then coated with a water-permeable polymer to produce the microcapsule preparation of this invention. For coating with the water-permeable polymer, organic solvents capable of dissolving polymers are used, such as ethanol, toluene, chloroform, methyl ethyl ketone, methylene chloride, isopropanol, cyclohexane, methanol, ethylene chloride, dimethylformamide, or ethyl acetate.

A plasticizer or a stabilizer, such as an antioxidant, may also be added in any amount. Examples of plasticizers include dibasic acid esters (phthalic acid esters, etc.), glycol esters, and fatty acid esters. Examples of antioxidants for stabilization include 2(3)-t-butyl-4-hydroxyanisol(BHA), 3,5-di-t-butyl-4-hydroxytoluene(BHT), tocopherol and tocopherol acetate.

When the water-permeable polymer is an acrylate-methacrylate copolymer, it is dissolved in methylene chloride or chloroform, the drug-resin complex then is added to and suspended in this solution. The resulting suspension is treated mechanically by the spray drying method to produce microcapsules; this can also be done by phase separation, a physico-chemical method. In this procedure the polymer is dissolved in a good solvent, a phase separating and anti-aggregating agent (chosen from polybutadiene, polydimethylsiloxane, methacryl polymer, etc.) is added to it in any amount, and a non-solvent is added while the solution is being stirred. Microcapsules can also be produced by a chemical method, i.e., the interfacial polymerization method. No matter which method is employed, it is preferable that the particle size of the sustained-release microcapsules thus obtained be from 5 to 1000 μm, or, more preferably, from 10 to 300 μm.

In a most preferred embodiment, the microcapsule preparation of this invention may be prepared as capsules in which the microcapsules are filled as well as a sustained-release suspension directly to be taken orally. The microcapsule preparation may also be combined with lactose, sucrose, corn starch, hydroxypropylcellulose, etc., to provide granules, powders or tablets.

The sustained-release preparation of this invention is characterized as follows: (1) a topiramate-ion exchange resin complex is produced in a continuous process in which a salt of topiramate is reacted in an aqueous solvent with an ion exchange resin to give the free form of topiramate, which can then be adsorbed by desired ion exchange resin. In this way, a complex is obtained which has adsorbed the topiramate in an amount nearer to the theoretical saturated adsorption amount than by the conventional production process based on equilibrium reaction; repetitive adsorption processes are not necessary, and drug loss is very small. In addition, it is possible to minimize the dose of the microcapsules making them economical, as well as easy to take; this dosage form design facilitates drug development. (2) In the present invention, the topiramate-resin complex can be produced with high efficiency because a higher topiramate adsorption rate is achieved as a result of the swelling of the ion exchange resin in the aqueous solvent, specifically a mixture of water and ethanol or methanol, to a higher degree than in the case in which water alone is used. (3) Since the topiramate-resin complex which has adsorbed the topiramate in a high concentration is coated with a water-permeable polymer which has a sustained-release property, the coats of microcapsules show neither cracking nor breaking (rupture) even when they are dispersed or suspended in a solvent. Dosage form designing can be done efficiently without using additives such as plasticizers in the coating materials as was previously done, thus ensuring the production of a preparation exhibiting an effective sustained-release property.

### EXAMPLES

The following examples further illustrates the present invention but should not be construed as in any way limiting its scope.

### Example 1

This example illustrates a method of administering a mania-alleviating amount of topiramate in a sustained-release formulation.

A dose of topiramate (e.g., 400 mg) in a sustained-release formulation is administered to the patient for at least four to six weeks. The Patient is monitored for a decrease in symptoms of mania as measured by the AMDS Mania-Depression Scale (Woggen, B., Int. Pharmacopsychiat., 14:228-242, 1979; Woggen, B., Int. Pharmacopsychiat., 14:325-337, 1979) or the Manic State Rating Scale (MSRS; Beigel, A., et al., Arch. Gen. Psychiat. 25:256-262, 1971; Cookson, J. C., et al., Neuropharmacology 18:1011-1013, 1979). The AMDS and MSRS will show clinical improvement in the symptoms of mania.

### Example 2

This example illustrates a method of administering a mania-alleviating amount of topiramate in a sustained-release formulation.

This experiment utilizes two test groups of patients. All of the patients are suffering from mania. The study is a randomized, double-blind, placebo controlled design. One treatment group receives a dose (e.g., 400 mg) of sustained-release topiramate, the other group receives a placebo. A dose of placebo or topiramate (e.g., 400 mg) in a sustained-release formulation is administered once a day to the patients for at least 4 weeks by the patients or medical professional. Efficacy is measured by the AMDS Mania-Depression Scale (Woggen, B., Int. Pharmacopsychiat., 14:228-242, 1979; Woggen, B., Int. Pharmacopsychiat., 14:325-337, 1979) or the Manic State Rating Scale (MSRS; Beigel, A., et al, Arch. Gen. Psychiat. 25:256-262, 1971; Cookson, J. C., et al., Neuropharmacology 18:1011-1013, 1979). Results are compared between the test group and the placebo group. Results will show greater alleviation of mania symptoms in the test group as compared to the control group. The difference in alleviation or amelioration of mania symptoms will be statistically significant.

### Example 3

This example illustrates the bio-equivalency with respect to the extent of absorption of the sustained-release topiramate as compared to administration of topiramate conventional immediate release tablet when administered as multiple daily doses.

In this study two groups of twenty human subjects are given a dose of 800 mg of topiramate either as 1) a single sustained-release dose, as embodied by the present invention, 2) conventional immediate release tablets of 200 mg administered four times a day or 400 mg tablets administered twice a day. Plasma concentrations of topiramate are measured serially to define the topiramate plasma concentration-time profile by published methods including gas chromatography (Riffitts, et al., A capillary gas chromatographic assay with nitrogen phosphorus detection for the quantification of topiramate in human plasma, urine and whole blood, J. Pharm. Biomed. Anal. 19:363-371, 1999) and HPLC (Gidal and Lensmeyrer, Therapeutic monitoring of topiramate: evaluation of the saturable distribution between erythrocytes and plasma of whole blood using an optimized high-pressure liquid chromatography method, Ther. Drug Monit. 21:567-576, 1999). The results will show that the plasma levels of both dose regimes produce essentially equivalent extent of absorption of topiramate, as measured by the area under the plasma topiramate concentration-time curve analysis. It is important to note, however, that when the sustained-release formulation of the present invention is given, the plasma level of topiramate will fluctuate less from time point to time point as compared to when the conventional immediate release tablet is administered as a multiple doses per day. (Percent fluctuation=100×(average Cmax−Cmin)/C average, where Caverage=AUC(0−τ)/τ, where τ is the dosing interval, AUC is area under the curve, Cmax is the maximum concentration and Cmin is the minimum concentration).

As is evident from the foregoing, the present invention contemplates novel compositions and treatment methods for utilizing a sustained-release formulation of topiramate. These novel compositions and methods allow for the circumvention of major limitations of past treatments thereby allowing for higher efficacy and more consistent dosing as compared to multiple dose regimes.

1. A sustained-release formulation comprising topiramate.

2. The sustained-release formulation of claim 1 wherein said formulation is an oral formulation selected from a group consisting of microcapsules, microgranules, tablets, capsules, aqueous suspension and slow-dissolve, fast disintegrating formulations administered transorally.

3. The sustained-release formulation of claim 1, wherein said topiramate is a pharmaceutically accepted salt.

4. A method of treatment comprising:
a) providing i) a patient displaying one or more symptoms of mania and ii) a sustained-release formulation comprising topiramate;
b) administering said formulation to said patient under conditions such that said one or more symptoms of said mania is reduced.

5. The method of claim 4 wherein, said topiramate is co-administered with an anti-psychotic.

6. A method of treatment, comprising:
a) providing, i) a patient exhibiting one or more symptoms of depression and ii) a sustained-release formulation comprising topiramate;
b) administering said formulation to said patient under conditions such that said one or more symptoms of said depression is reduced.

7. The method of claim 7 wherein, said topiramate is co-administered with an anti-depressant.

8. A method of treatment, comprising:
a) providing, i) a patient exhibiting one or more symptoms of bipolar disorder and ii) a sustained-release formulation comprising topiramate;
b) administering said formulation to said patient under conditions such that said one or more symptoms of said bipolar disorder is reduced.

9. The method of claim 10 wherein, said topiramate is co-administered with an anti-psychotic.
